(19) United States
(12) Patent Application Publication
(10) Pub. No.: US 2018/0126189 A1
(43) Pub. Date: May 10, 2018

(54) Precise Proton Positioning Method for Proton Therapy Treatment, and Proton Therapy Treatment Method

(71) Applicant: The Trustees of Princeton University, Princeton, NJ (US)

(72) Inventors: Philip EFTHIMION, Bedminster, NJ (US); Kenneth W. HILL, Plainsboro, NJ (US); Lan GAO, Princeton, NJ (US); Manfred BITTER, Princeton, NJ (US); Dale MEADE, Princeton, NJ (US); Brian Francis KRAUS, Princeton, NJ (US)

(21) Appl. No.: 15/601,215

(22) Filed: May 22, 2017

Related U.S. Application Data
(60) Provisional application No. 62/340,584, filed on May 24, 2016.

(51) Int. Cl. A61N 5/10; A61L 31/02; A61L 31/14

(52) U.S. Cl. CPC A61N 5/1049; A61N 5/1067; A61B 2090/392; A61L 31/022; A61L 31/14; A61N 5/1077

(57) ABSTRACT

A system/apparatus and a positioning method for the determination of the delivery of protons within a mammalian body or inanimate medium, as well as a treatment method which comprises the delivery of protons to a mammalian body. The system/apparatus necessarily comprise the use of a detector which is placed in the proximity of the body or medium which is responsive to a signal emitted from a fiducuial marker which his positioned within or in the near proximity of target tissue within the body or target volume within the medium. A signal emitted from the fiducial marker is responsive to the impingement of protons thereon received from a controlled proton beam, the signal may be used to modify the subsequent operative characteristics of the source of protons.

This application claims the priority benefit of U.S. Ser. No. 62/340,584 filed 24 May 2016, and herein incorporates by reference the entirety of that prior application.

This invention was made with US Government Support under Grant No. DE-AC02-09CH11466 awarded by the Department of Energy. The US Government has certain rights in the invention.

The present invention relates to a system and/or devices and a positioning method for the determination of the delivery of protons within a mammalian body, as well as a treatment method which comprises the delivery of protons to a mammalian body.

The system and/or method of the present invention is useful in the treatment of tumors within a mammalian body. A tumor is an abnormal mass of tissue. Tumors are either benign or malignant. A benign tumor grows locally, but does not spread to other parts of the body. Benign tumors cause problems because of their spread, as they press and displace normal tissues. Benign tumors are dangerous in confined places such as the skull. A malignant tumor is capable of invading other regions of a mammalian body. Metastasis is cancer spreading by invading normal tissue and spreading to distant tissues within the body. Tumors are type of tissue which are treated by one or more forms of radiation therapy used in the treatment of mammalian bodies.

Several forms of radiation therapy exist for cancer treatment including: brachytherapy, traditional electromagnetic X-ray therapy, and proton therapy. Brachytherapy is radiation therapy using radioactive sources implanted inside the body. In this treatment, an oncologist implants radioactive material directly into the tumor or very close to it within the body. Electromagnetic radiation includes treatment using X-rays and gamma rays. An X-ray is high-energy, ionizing, electromagnetic radiation that is used at low doses to diagnose disease or at high doses to treat cancer. An X-ray or Röntgen ray is a form of electromagnetic radiation with a wavelength in the range of 10 to 0.01 nanometers (nm), corresponding to frequencies in the range of 30 PHz to 30 EHz. X-rays are longer than gamma rays and shorter than ultraviolet rays. X-rays are primarily used for diagnostic radiography. X-rays are a form of ionizing radiation and can be dangerous. Gamma rays are also a form of electromagnetic radiation and are at frequencies produced by sub-atomic particle interactions, such as electron-positron annihilation or radioactive decay. In the electromagnetic spectrum, gamma rays are generally characterized as electromagnetic radiation having the highest frequency, as having highest energy, and having the shortest wavelength, such as below about 10 picometers. Gamma rays consist of high energy photons with energies above about 100 keV. X-rays are commonly used to treat cancerous tumors. However, X-rays are not always best for treatment of cancerous tissue as X-rays deposit their highest dose of radiation near the surface of the targeted tissue and delivery exponentially less radiation as they penetrate into the tissue. This results in large amounts of radiation being delivered outside of the tumor. Gamma rays have similar limitations to those described with reference to X-rays.

A further form of cancer therapy uses protons. Proton therapy systems typically include: a beam generator, an accelerator, and a beam transport system to move the resulting accelerated protons to a treatment room, more frequently a plurality of treatment rooms where the protons are delivered to a tumor in a patient's body, viz. a mammalian body. Proton therapy works by aiming energetic ionizing particles, such as protons accelerated with a particle accelerator, onto a target tumor. These particles damage the DNA of cells, ultimately causing their death. Cancerous cells, because of their high rate of division and their reduced ability to repair damaged DNA, are particularly vulnerable to attack on their DNA. Energy lost by charged particles, such as protons, is inversely proportional to the square of their velocity, which explains the peak energy absorption occurring just before the particle comes to a complete stop. Protons beams lose very little energy going through the body until its velocity is sufficiently low that it deposits all of its energy in a short distance, a few inches. Proton and other charged particle beams, unlike x-ray and gamma rays, do not damage tissue entering the body until it slows down and stops. Then it deposits all of its energy. Consequently, protons and other charged particle beams are a better cancer therapy than x-rays and gamma rays because it creates far less collateral damage. Such may thus be used advantageously. All protons of a given energy have a certain range, defined by the Bragg peak, and the dosage delivery to tissue ratio is maximum over just the last few millimeters of the particle's range. Delivery of protons can be controlled to thus minimize damage to tissue (or other mammalian body parts) and to effectively treat a tumor within the mammalian body. The penetration depth depends on the energy of the particles, which is directly related to the speed to which the particles were accelerated by the proton accelerator. The speed of the proton is adjustable up to the maximum rating of the accelerator. It is therefore possible to direct the proton beam so to deliver these particles at the very depth in the tissues where the tumor is situated. Tissues (or locations) situated before the Bragg peak receive some reduced dose and tissues situated after the peak receive none.

Various devices and systems may be used in proton therapy systems and methods for proton treatment of a mammalian, e.g., human, body. Such are described in the prior art, e.g., in U.S. Pat. No. 8,129,699 B2 to Balakin as well as U.S. Pat. No. 7,953,205 B2 to Balakin. Certain proton beam therapy systems are known from U.S. Pat. No. 4,870,287 for a “Multi-Station Proton Beam Therapy System” which describes a proton beam therapy system for selectively generating and transporting proton beams from a single proton source and accelerator to a selected treatment room of a plurality of patient treatment rooms. U.S. Pat. No. 7,262,424 for “Particle Beam Therapy System”, describes a particle beam therapy system that uses information from treatment rooms to control delivery of the ion beam to one of a plurality of treatment rooms. U.S. Pat. No. 7,227,161 for “Particle Beam Irradiation Apparatus, Treatment Planning Unit, and Particle Beam Irradiation Method” describe a particle beam irradiation apparatus have a scanning controller that stops output of an ion beam, changes irradiation position via control of scanning electromagnets, and reinitiates treatment based on treatment planning information. U.S. Pat. No. 7,060,997 for “Particle Therapy System Apparatus”, and U.S. Pat. No. 6,936,832 for “Particle Therapy System Apparatus” each describe a particle therapy system having a first steering magnet and a second steering magnet disposed in a charged particle beam path after a synchrotron that are controlled by first and second beam position monitors. U.S. Pat. No. 6,984,835 for “Irradiation Apparatus and Irradiation Method” describe an irradiation method having a large irradiation filed capable of uniform dose distribution, without strengthening performance of an irradiation field device, using a position controller having overlapping area formed by a plurality of irradiations using a multileaf collimator. The system provides flat and uniform dose distribution over an entire surface of a target. U.S. Pat. No. 6,903,351 for Charged Particle Beam Irradiation Equipment Having Scanning Electromagnet Power Supplies” discloses power supply for applying a voltage to a scanning electromagnet for deflecting a charged particle beam and a second power supply without a pulsating component to control the scanning electromagnet more precisely allowing for uniform irradiation of the irradiation object. Described in U.S. Pat. No. 6,859,741 for a “Device and Method for Adapting the Size of an Ion Beam Spot in the Domain of Tumor Irradiation” is a method and apparatus for adapting the size of an ion beam in tumor irradiation. Quadrupole magnets determining the size of the ion beam spot are arranged directly in front of raster scanning magnets determining the size of the ion beam spot. The apparatus contains a control loop for obtaining current correction values to further control the ion beam spot size. U.S. Pat. No. 6,717,162 discusses a method for treating a tumor using a proton beam, where a particle beam is used to produce a narrow spot whose sweeping speed and intensity are simultaneously varied. U.S. Pat. No. 5,986,274 for a “Charged Particle Irradiation Apparatus and an Operating Method Thereof” discloses a charged particle irradiation apparatus capable of decreasing a lateral dose falloff at boundaries of an irradiation field of a charged particle beam using controlling magnet fields of quadrupole electromagnets and deflection electromagnets to control the center of the charged particle beam passing through the center of a scatterer irrespective of direction and intensity of a magnetic field generated by scanning electromagnets. Formation of particle beams is disclosed in U.S. Pat. No. 5,760,395 for a “Method and Apparatus for Laser Controlled Proton Beam Radiology”, and in U.S. Pat. No. 5,177,448 for “Synchrotron Radiation Source With Beam Stabilizers”. Injection of charged particles is disclosed in U.S. Pat. No. 4,870,287 for an “Accelerator System”, and in U.S. Pat. No. 5,789,875 for a “Circular Accelerator, Method of Injection of Charged Particle Thereof, and Apparatus for Injection of Charged Particle Thereof”. In U.S. Pat. No. 7,259,529 for “Charged Particle Accelerator” is described a charged particle accelerator having a two period acceleration process with a fixed magnetic field applied in the first period and a timed second acceleration period to provide compact and high power acceleration of the charged particles. In U.S. Pat. No. 5,168,241 is described an acceleration cavity having a high frequency power source and a looped conductor operating under a control that combine to control a coupling constant and/or de-tuning allowing transmission of power more efficiently to the particles. U.S. Pat. No. 6,087,670 for a “Synchrotron Type Accelerator and Medical Treatment System Employing the Same” discloses a synchrotron accelerator having a high frequency applying unit arranged on a circulating orbit for applying a high frequency electromagnetic field to a charged particle beam circulating and for increasing amplitude of betatron oscillation of the particle beam to a level above a stability limit of resonance. U.S. Pat. No. 5,285,166 for Method of Extracting Charged Particles from Accelerator, and Accelerator Capable Carrying Out the Method, by Shifting Particle Orbit” describes a method of extracting a charged particle beam, in which an equilibrium orbit of charged particles maintained by a bending magnet and magnets having multipole components greater than sextuple components is shifted by a constituent element of the accelerator other than these magnets to change the tune of the charged particles. U.S. Pat. No. 6,800,866 for an “Accelerator System and Medical Accelerator Facility”, discusses an accelerator system having a wide ion beam control current range capable of operating with low power consumption and having a long maintenance interval. U.S. Pat. No. 6,859,741 for “Device and Method for Adapting the Size of an Ion Beam Spot in the Domain of Tumor Irradiation”, describes a method and apparatus for adapting the size of an ion beam in tumor irradiation. Quadrupole magnets determining the size of the ion beam spot are arranged directly in front of raster scanning magnets determining the size of the ion beam spot. The apparatus contains a control loop for obtaining current correction values to further control the ion beam spot size. U.S. Pat. No. 6,717,162 for “Method for Treating a Target Volume with a Particle Beam and Device Implementing Same”, discusses a method of producing from a particle beam a narrow spot directed toward a target volume, characterized in that the spot sweeping speed and particle beam intensity are simultaneously varied. U.S. Pat. No. 6,799,068 for a “Method For Verifying the Calculated Radiation Dose of an Ion Beam Therapy System”, describes a method for the verification of the calculated dose of an ion beam therapy system that comprises a phantom and a discrepancy between the calculated radiation dose and the phantom. U.S. Pat. No. 7,274,025 for a “Detector for Detecting Particle Beams and Method for the Production Thereof” discloses a detector and a method of making the detector. The detector comprises a crystalline semi-conductor diamond plate and a aluminum metal coating arranged on a ceramic plate substrate. U.S. Pat. No. 5,039,867 for a “Therapeutic Apparatus”, describes a method and apparatus for positioning a therapeutic beam in which a first distance is determined on the basis of a first image, a second distance is determined on the basis of a second image, and the patient is moved to a therapy beam irradiation position on the basis of the first and second distances.

While the above apparatus, systems and methods provide various benefits and technical features, there still remains an urgent need in the art for improved apparatus, systems and method for proton therapy.

In a first aspect of the invention there is provided a positioning method for the determination of the delivery of protons within a mammalian body. The positioning method is novel, and allows for determining the end point of the proton beam in proton therapy treatment procedures. A fiducial marker is implanted with the tumor to align the patient with respect to the path of the beam to make sure the beam will enter the tumor and not miss it. This is verified by a CT scan, or other imaging means, that will observe and/or verify the location of the fiducial marker. The presence of the implanted fiducial marker allows for improved control and delivery of protons to target tissue. Based on the precision measurements, proton beam characteristics used for treatment of tissue within a mammalian body can be accurately identified, allowing the operators of a proton accelerator to adjust the proton beam correspondingly so that it targets the delivery of charged particles to the locus (or volume) of target tissue, e.g, a tumor. Preferably minimal discharge of energy from the charged particles occurs to the exterior of the target tissue.

In a second aspect of the invention there is provided a further positioning method for the determination of the delivery of protons within a mammalian body. The positioning method is novel, and allows for determining the end point of the proton beam in proton therapy treatment procedures. According to this method, a fiducial marker is implanted near the boundary or edge of target tissue, e.g. a tumor in addition to a fiducial marker implanted within the target tissue. The use of such spaced apart fiducial markers allows for improved control and delivery of protons to target tissue. Based on the precision measurements, proton beam characteristics used for treatment of tissue within a mammalian body can be accurately identified, allowing the operators of a proton accelerator to adjust the proton beam correspondingly so that it targets the delivery of charged particles to the locus (or volume) of target tissue, e.g, a tumor. Preferably minimal discharge of energy from the charged particles occurs to the exterior of the target tissue.

A third aspect of the invention provides an improved proton therapy treatment method, which is advantageously used in the targeted delivery of charged particles, especially protons from a proton source, to a desired locus (or volume) within a mammalian body. The treatment method is advantageously employed in a therapeutic regimen for controlling, diminishing or eradicating the presence of undesired tissues, especially cancerous tissues or tumors within a mammalian body. The improved proton therapy treatment method provides for targeted delivery of charged particles to the locus (or volume) of target tissue, with minimal discharge of energy from the charged particles occurring within the exterior of the target tissue. The method allows for diagnosing the end point of the proton beam in proton therapy treatment procedures. Based on precision measurements, proton beam characteristics used for treatment of target tissue can be accurately identified, allowing the operators of a proton accelerator to adjust the proton beam correspondingly so that it more accurately delivers the protons to the desired locus. According to a particularly preferred embodiment of this third aspect, the end point of a proton beam coincides with or occurs within the target tissue, which minimizes the delivery of protons outside of the desired locus.

A further aspect of the invention is an apparatus/system useful in providing an improved positioning method for the determination of the controlled delivery of protons within a mammalian body.

A further aspect of the invention is an apparatus/system useful in providing an improved positioning method for the determination of the controlled delivery of protons within a three dimensional space which is outside of a mammalian body.

A further aspect of the invention is an improved positioning method for the determination of the controlled delivery of protons within a three dimensional space which is outside of a mammalian body.

A yet further aspect of the present invention is an apparatus/system useful in providing an improved proton therapy treatment method, which method provides for the targeted delivery of charged particles, especially protons from a proton source, to a desired locus (or volume) within a mammalian body, e.g. to target tissue, or tumor, within a mammalian body.

These and further aspects of the invention will become more apparent from the following specification and accompanying drawings.

The system/apparatus and methods of the present invention may be used to determine whether the proton beam (and particularly the end point of a proton beam) is present in specific locations in the mammalian body, and enables the operators of the proton accelerator to adjust the proton beam until it precisely targets the desired region of tissue. The apparatus and methods of the present invention are superior to many other known art apparatus and methods, which methods generally have larger position uncertainties because they rely on either (1) calculations of beam penetration as a function of beam energy, which can have larger uncertainties due to variations of tissue types in the beam path; (2) positron-electron tomography (PET), which has larger position uncertainties; or (3) imaging of gamma rays produced by the proton beam, which has a smaller cross section, have weaker signals and larger uncertainties.

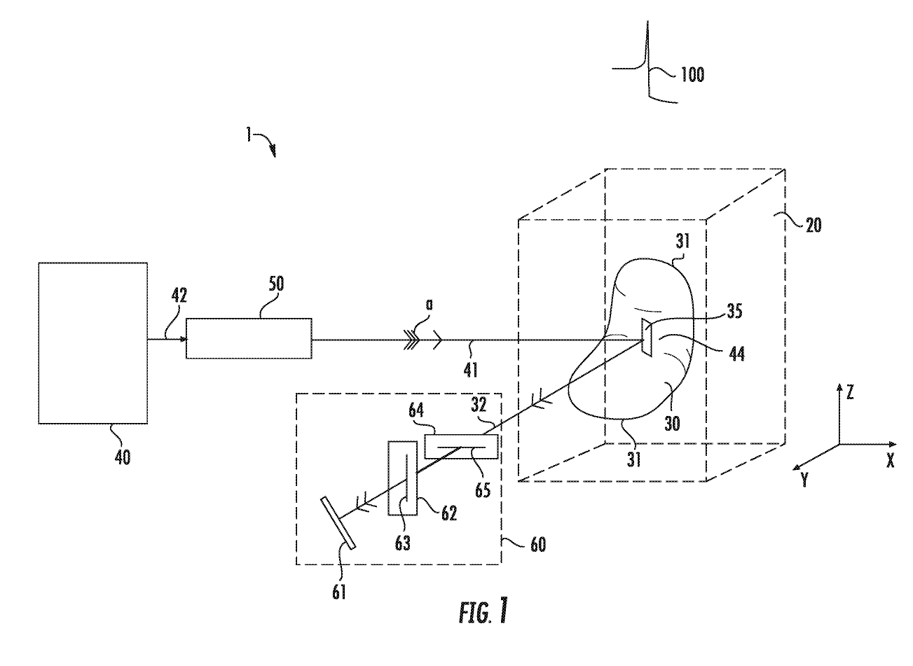

FIG. 1 depicts a schematic view of a proton therapy treatment apparatus and system (apparatus/system), illustrating the use of the apparatus and system according to a method taught herein.

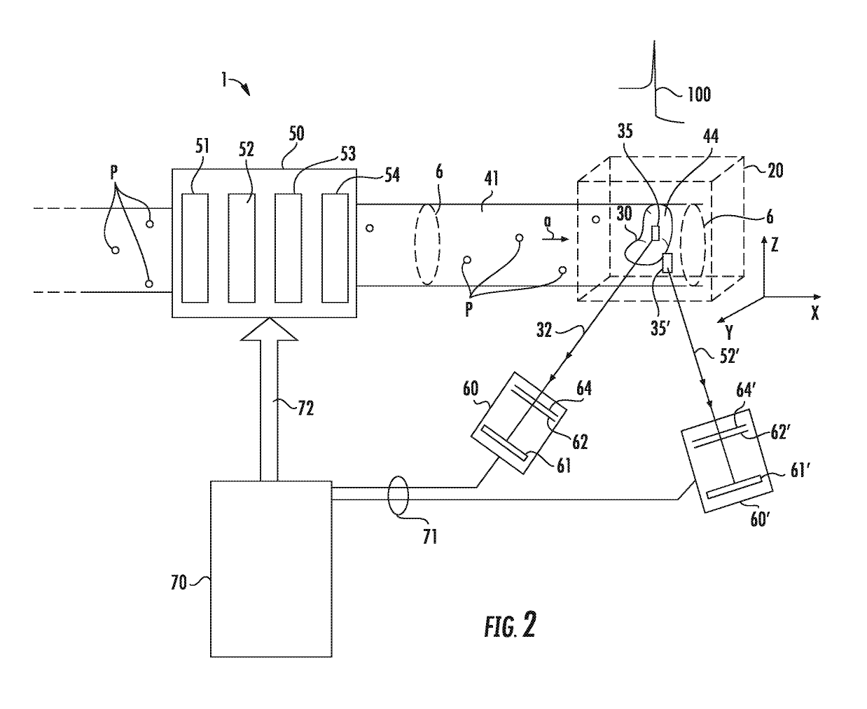

FIG. 2 depicts a schematic view of a proton therapy treatment apparatus and system, illustrating the use of the apparatus and system according to a method taught herein, which utilizes a plurality of implanted fiducial markers.

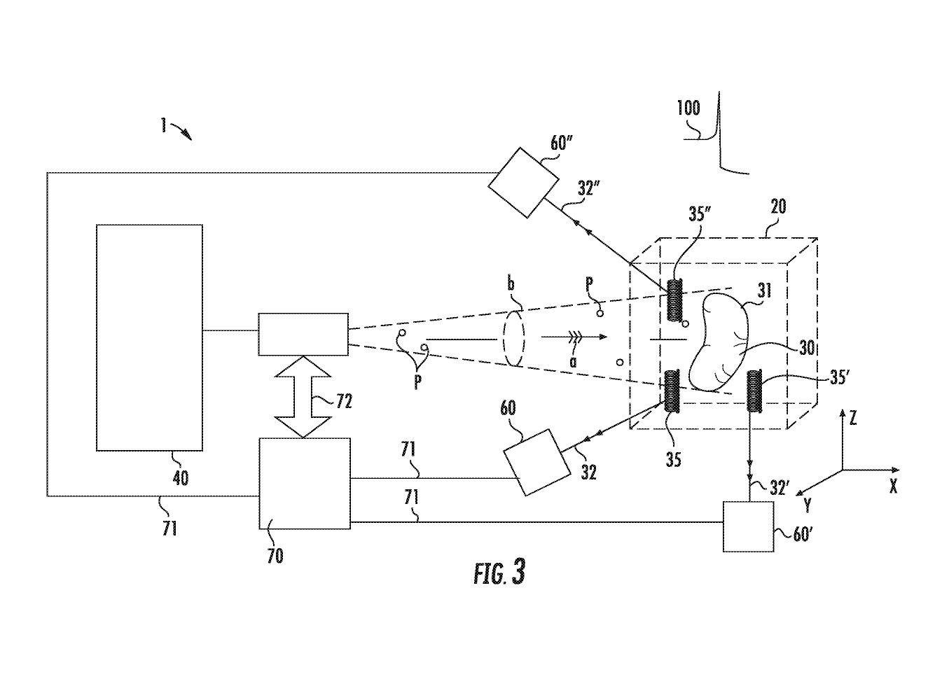

FIG. 3 depicts a schematic view of a proton therapy treatment apparatus and system, illustrating the use of a the apparatus and system according to a further method taught herein, which utilizes a plurality of implanted fiducial markers.

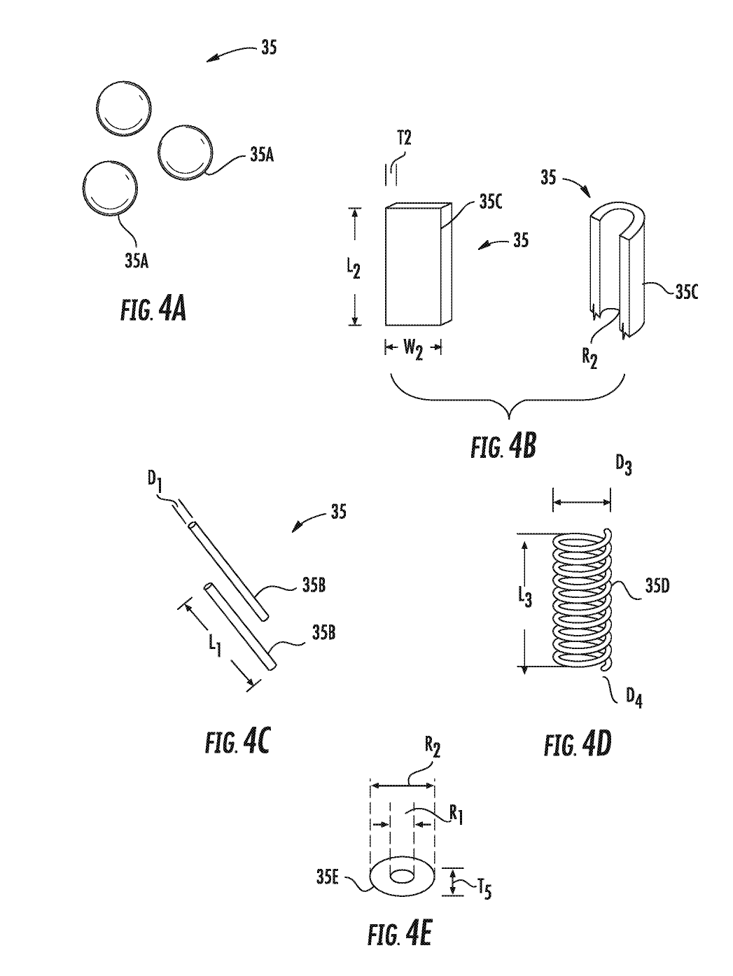

FIGS. 4A, 4B, 4C, 4D and 4E depict various embodiments of fiducial markers, useful with the proton therapy treatment apparatus and system and the proton treatment therapy methods taught herein. FIG. 4A depicts a sphere shaped fiducial markers. FIG. 4B depicts plate shaped fiducial markers. FIG. 4C depicts rod-shaped fiducial markers. FIG. 4D depicts helically shaped fiducial markers. FIG. 4E depicts a toroidal shaped fiducial marker.

The methods taught herein may be used with existing apparatus and treatment methods currently known to the art. A method of the invention is useful in the treatment of tumors, target tissue or volumes present within a mammalian body. A preferred mammalian body is human body. The preferred human body is on which contains a tumors, target tissue or volumes present within, which is to be treated with protons; such may be interchangeably referred to herein as a “patient”, or a “patient's body”.

The methods taught herein may be used with a non-mammalian body, viz. to deliver protons a/o a proton beam to an inanimate three-dimensional space/volume, or an inanimate “medium”.

The method taught herein adds to known-art treatment methods and known-art apparatus but is novel thereover. The novel method of the invention requires the use of a fiducial marker and a responsive sensor, which may be used to increase the accurate delivery of protons to a target tissue within a mammalian body, and/or a patient. The fiducial marker is an article or material which his positioned within or in the near proximity of target tissue within the patient. The fiducial marker is advantageously embedded within target tissue, such as a tumor or cancerous tissue which is also present in a patient. The fiducial marker may be embedded by non-surgical techniques or may be embedded or inserted by surgical means to ensure appropriate placement. The fiducial marker necessarily comprises a material which is responsive to the impingement of protons thereon.

The method (and apparatus) taught herein are also useful for the determination of the controlled delivery of protons within a three dimensional space which is outside of a mammalian body.

Thus, use of an embedded fiducial marker provides for an effective method for determining or validating the presence of photons being delivered to the fiducial marker and thus concurrently to the near proximity thereof, namely in the target tissue. The response of a fiducial marker may be any response which may be sensed by an appropriate sensor or detector device or apparatus. The response from the fiducial marker may provide a qualitative and/or quantitative indication of the protons and/or energy being delivered to the locus of the embedded fiducial marker. The monitoring of the response from an embedded fiducial marker provides for a high degree of control over the specific delivery and placement of delivered photons within a mammalian body. As is known, protons scatter less easily than X-rays or gamma rays, and may be more accurately focused on the shape of targeted tissue, e.g. a tumor, without imparting much lateral damage to surrounding tissue. Such a property is highly advantageous in the treatment of patients as parts of the mammalian body in the proximity of the target tissue being treated receives little or no protons. Thus, the maximum effect of delivered protons from a proton source, such as a particle beam from a proton source, may be effectively used to provide improved therapies and treatment methods particularly of tumors, especially cancerous tumors within a mammalian body, while minimizing damage to surrounding tissue. As all protons of a given energy have a certain range, defined by the Bragg peak, and the dosage delivery to tissue ratio is maximum over just the last few millimeters of the particle's range the improved targeted delivery of protons provided by the apparatus, systems and methods of the present invention allows for reduced likelihood of damage to tissue (or other mammalian body parts) in the locus of target tissue, while effectively treating the target tissue within the patient's body. Such improved targeting using the apparatus, systems and methods of the present invention allows for effective therapeutic treatment of target tissue with lower treatment dosages as the delivery of proton particles is more accurate than previously possible. Thus, with such improved targeting it now becomes possible to more precisely direct the proton beam so to deliver proton particles at the very depth and position within a mammalian body wherein target tissue is located. And as it is a known characteristic that tissues situated before the Bragg peak receive some reduced dose and tissues situated after the peak receive none, and thus the proton beam end point generally corresponds to the Bragg peak, and its position particularly where it relates to a patient's body. The improved targeting provides for maximizing the delivery of the proton particles within the target tissue, which thus frequently allows for a relatively reduced dosage (or reduced number of dosages) to effectively treat the target tissue, as compared to an otherwise identical proton therapy treatment method which does not utilize a fiducial marker and an appropriate sensor or detector device or apparatus. The improved targeting preferably enables the delivery of protons from a proton source, e.g. a cyclotron which emits a controllable proton beam which has an end point corresponding to the Bragg peak, and which end point is directed to be within the target tissue, or which Bragg peak coincides with or which occurs within the target tissue.

The fiducial marker may be any device, article or material which emits a signal or material in response to the impingement of protons upon its surface and/or within its volume or mass. Non-limiting examples of materials which may be used as a fiducial marker, or used in the construction of a fiducial marker article or device include one or more of: gold, gold alloys, platinum, silver palladium, zirconium, molebdenum, and combinations or mixtures (e.g. alloys) thereof.

Optionally but preferably the fiducial marker is made of or at least upon its exterior thereof includes a biocompatible material to thus minimize the rejection of the fiducial marker which is embedded or otherwise located within target tissue or within the near proximity of target tissue present in a mammalian body.

The dimensions or size of the fiducial marker are not critical to the operation of the systems and method of the invention, as it is only required that the fiducial marker provide or emit a signal or emit a material which may be sensed by the responsive sensor. Such may be, for example be particles or radiation which is sensed by one or more responsive sensors placed in the proximity of a patient's body being treated with protons. Such are preferably X-rays. Advantageously fiducial markers have minimum dimension in the range of about 0.05-5 mm, preferably about 0.2-0.5 mm, and have a maximum dimension in the range of about 0.5-5 mm, preferably about 2.5-5 mm. The fiducial markers may be solid materials, which conform to a regular geometric shape, e.g, sphere, cube, cuboid, rectangular shape, loop, ring, helix, coil, or may be of any other shape. In one embodiment the fiducual markers comprise one or more particulates of a comminuted suitable material, e.g. a metal or metal alloy which individual particles may have the same or different dimensions. In another and preferred embodiments the fiducial markers are coils formed of a bar or wire of a suitable material, e.g. a metal or metal alloy, wherein the wire or bar has cross-section of between about 0.05-5 mm, preferably about 0.5-1.25 mm, and the bar or wire has a helical configuration with an external diameter of the coil of from about 0.25-10 mm, preferably about 0.5-1 mm, and a length between the two ends of the coil being from about 0.5-10 mm, preferably about 3-5 mm. In another embodiment the fiducial markers are generally spherical in configuration, and have an external diameter of from about 0.25-2.5 mm, preferably about 0.5-1 mm. Other shapes, configurations and dimensions of the fiducial marker may also be used, and fall within the scope of the present invention.

While one fiducial marker is usually sufficient, the method and systems of the present invention may benefit from the concurrent use of two of more fiducial markers.

The fiducial markers may be embedded or otherwise provided to a position within or upon a mammalian body, such as within a target tissue or in the proximity of a target tissue. The fiducial markers may also be provided to a volume or a space, e.g. cavity, within a mammalian body. Preferably the fiducial marker is positioned or embedded within, or is immediately adjacent to or at least, in part, contacts target tissue of the patient's body.

One or more fiducial markers may be placed within the target tissue, or may be placed at or near the exterior of the target tissue, viz, the boundary of the target tissue, or may be placed elsewhere within or upon a mammalian body. The usual markers may be placed by any conventional means, such as by surgical means. Such may be invasive and require surgical procedure, but are not necessarily so particularly if a crucial markers to be placed in an existing body cavity, e.g. cervix, rectum. Advantageously, one surgical procedure may be the use of a relatively wide bore cannula which may be inserted through the epidermis and into the interior, or in the near proximity of target tissue, wherein the fiducial marker is delivered via the central bore of the cannula to a desired position, and thereafter the cannula is withdrawn. The precise placement of one or more fiducial markers within a patient's body can be facilitated by the use of other diagnostic or imaging techniques, that is to say radiographic techniques, e.g., x-rays, CAT scan, or Mill scanning and/or imaging methods. The relative dimensions, orientation, and position of target tissue within a patient's body can also be determined by such other diagnostic or imaging techniques, which can be utilized to provide a three-dimensional map and/or to provide a three-dimensional relative positioning of target tissue intended to be treated to provide the proton therapy methods described herein. Predetermination of the relative dimensions, orientation, position of target tissue within a patient's body greatly facilitates the precise delivery of, or insertion of one or more fiducial markers within a patient's body, especially prior to the delivery of a proton beam to the locus of the fiducial markers, which are preferably within, or near proximity to the target tissue. Thus, the relative positioning of one or more fiducial markers, but preferably where two or more fiducial markers are concurrently present allows for a number of reference points to be established within a three-dimensional geometric space, with each reference point being represented by the position of a fiducial marker.

Subsequent to proton therapy treatment, the fiducial markers may be allowed to remain within a patient's body, or may be removed from the patient's body.

### Responsive Sensor:

The responsive sensor may be any article, apparatus or device which is effective in sensing or detecting particles or the a signal emitted from the fiducial marker present in the mammalian body, which release is caused by the impingement of protons from the proton source on the fiducial marker. Preferably the fiducial markers emit x-rays when bombarded with protons, or when protons from a proton source impinge upon the fiducial marker.

In preferred embodiments the responsive sensor is an x-ray detector, or may be a pixel array detector which provides for selecting and counting x-ray photons of a particular energy emitted by the one or more fiducial markers. In certain preferred embodiments the x-ray detectors are responsive to a range of about 1-500 keV, more preferably about 15-100 keV. When the proton beam collides with the fiducial, it emits x-rays that can be detected by the x-ray detector. A fiducial material is selected that emits x-rays of sufficiently high energy that they pass through the body and are detected by the x-ray detector.

The responsive sensor (or responsive sensors) may each include a slit or a set of slits which are used in limiting the operation of a responsive sensor to only receive particles or sense signals along a limited essentially two dimensional plane, or along a particular essentially linear direction so that an individual responsive sensor may be responsive so to detect particles or a signal emitted from a single fiducial marker positioned within a mammalian body. Thus the use of two or more such responsive sensors which each include a slit or a set of slits may be used to detect receive particles or sense signals only from a specific location. Data collected from each responsive sensor may be used to validate the position of the end point of the proton beam, and can be used to adjust the position of the proton beam entering the mammalian body so that the delivery to the target tissue is optimized. In such a manner, the proton beam is administered to a patient in a small dose and the x-ray measurements are evaluated, as the delivery of the protons impinging upon embedded fiducial markers releases particles or a detectible signal, e.g. x-rays. Based upon the x-ray measurements received by the one or more responsive sensors a new optimal energy for the proton beam to be absorbed by the tumor is estimated, and a further (subsequent) dose of protons may be administered to the patient. The x-ray measurements may also be used to provide information indicative of the location of the end point of the proton beam, and whether the Bragg peak is within the target tissue; if not, the information may be used to determine adjustments to the direction and optionally the intensity of the proton beam in order to ensure more accurate delivery of the end point to the target tissue. Computer simulation codes and/or software may be used to control the above process.

One or more responsive sensors are placed in the in the proximity of a patient's body being treated with protons. Preferably the patient's body is immobilized during delivery of protons to the patient's body. The use of two or more such responsive sensors may be preferred in certain embodiments especially wherein such are separated by a physical distance, or a line. The use of three or more physically spaced apart responsive sensors is also advantageous as providing more data concerning the position of the end point of the proton beam within the mammalian body. As the location of the end point of the proton beam is a function of the beam energy and can be moved by adjusting the beam energy; such corresponds to the Bragg peak. When the proton beam, and preferably the end point of the proton beam impinges or upon a fiducial marker, one or more responsive sensors may receive a particle or sense a signal caused by the action of protons on the fiducial marker. In a preferred embodiment, x-rays are generated and released from the fiducial marker. Thus, wherein a fiducial marker is inserted into a target tissue, especially at or near the center thereof, the detection of emitted x-rays by one or more responsive sensors validates that the endpoint of the proton beam is within the target tissue. When the fiducial marker is exposed to impinging photons or other charged particles, the particles or signals released from it can be determined by one or more suitable responsive sensors, and based on the nature of the particles or signals from the fiducial marker, and its relative position with respect to the volume of the target tissue and boundary thereof, the position of the end point (or Bragg peak) relative to the target tissue and boundary can be ascertained, e.g., by estimation or calculation, either to validate the presence of protons impinging on the fiducial marker, and preferably to validate that the Bragg peak occurs within the target tissue, or alternately and if necessary, to subsequently modify the direction of and/or adjust the energy of the proton beam to be delivered in a successive dose, or successive treatment of the patient.

In an alternative embodiment, at least two fiducial markers are present within, or in the near proximity of target tissue within a patient's body. In this further embodiment, at least one of the fiducial markers is placed within the mass, volume, or space occupied by the target tissue present within the patient's body, and at least one further fiducial marker is placed at, or near the exterior of the target tissue. Thus these at least two fiducial markers are physically spaced apart, and the distances between them can be established. When exposed to impinging photons (or other particles), the particles or signals released from each of the individual fiducial marker can be determined by suitable responsive sensors, and based on the energy of the particles or signals from each individual fiducial marker, and their relative position with respect to each other, and with respect to the target tissue and boundary thereof, the position of the end point (or Bragg peak) relative to the target tissue and boundary can be ascertained, e.g., by estimation or calculation. This information can be used, if necessary to subsequently modify the direction of and/or adjust the energy of the proton beam to be delivered in a successive dose, or successive treatment of the patient. According to a preferred embodiment the fiducial has is carefully selected so it emits photons (x-rays) of high enough energy that they will pass out of the body and be detected. It is nonetheless to be understood that the principles of the invention are fully operative and may be used in conjunction with particles than protons other than protons as described herein.

Alternatively none of the fiducial markers are placed within the target tissue, but are placed at or near the exterior of the target tissue, viz, the boundary of the target tissue. In this manner, one or more responsive sensors can sense particles or signals released were delivered from the two or more fiducial markers; the physical spacing between the fiducial markers thus provides an improved validation that the position and the width of the proton beam being delivered to the patient's body is sufficiently large to encompass the confines of the target tissue. Also, the nature of the particles or signals from each individual fiducial markers, and their relative position with respect to each other, and with respect to the target tissue and boundary thereof, the position of the end point (or Bragg peak) relative to the target tissue and boundary can be ascertained, e.g., by estimation or calculation. This information can be used, if necessary, to subsequently modify the direction of and/or adjust the energy of the proton beam to be delivered in a successive dose, or in successive treatment of the patient.

In a yet further alternative embodiment, three or more fiducial markers are present within, or in the near proximity of target tissue within a patient's body. Preferably at least one of these three or more fiducial markers are present within the boundaries of the target tissue, and are preferably physically present within the interior of the target tissue, preferably at or near the center thereof, whereas the remaining fiducial markers may be present at or near the exterior of the target tissue. Alternatively none of the three or more fiducial markers are actually present within the interior of the target tissue but rather are present at or near the exterior of the target tissue, viz. the boundary of the target tissue or in the proximate locus of the target tissue. In either instance, the fiducial markers are responsive to impinging protons and as described above, the proton beam, and preferably the end point of the proton beam impinges or upon a fiducial marker, one or more responsive sensors may receive a particle or sense a signal caused by the action of protons on the fiducial markers. Preferably the responsive sensors may receive a particle or sense a signal caused by the action of protons on the fiducial markers when the Bragg peak of the protons is coincident with the target tissue of a patient being treated. The nature of the particles or signals from each individual fiducial markers, and their relative position with respect to each other, and with respect to the target tissue and boundary thereof, the position of the end point (or Bragg peak) relative to the target tissue and boundary can be ascertained, e.g., by estimation or calculation. This information can be used, if necessary, to subsequently modify the direction of and/or adjust the energy of the proton beam to be delivered in a successive dose, or in successive treatment of the patient.

The above fiducial markers may be used with conventional proton therapeutic methods and apparatus, including those known to the prior art as well as techniques and apparatus which may become known to the art subsequent to the filing date of this patent application.

The dosage necessary to control, reduce or eradicate particular target tissues, especially tumors within a mammalian body, may vary widely and in great part depends upon the nature of the target tissue (e.g., tumor), is location within a mammalian body, and the health of the patient being treated. A single treatment or a single dosage may be used to control, reduce or eradicate particular target tissues but more frequently a treatment regimen requires the multiple treatments in which an individual dosage of protons is to be delivered to the target tissue. Thus repetitive treatments, and multiple dosing are typically necessary. The apparatus/system and methods of the invention may be used for both single, or multiple dosing treatment regimens.

While not intending to be limiting, an exemplary single dosage is between about 1-10 minutes, preferably about 5 minutes (+/−1.5 minutes) irradiation at a proton beam current of 50 nA. Other dosages may be used, and the apparatus/system and methods of the invention may be used for any type of treatment regimen, including those which deliver single or multiple dosing treatments as part of the regimen.

The dosage necessary to control, reduce or eradicate particular target tissues, especially tumors within a mammalian body, may conceivably be reduced by use of the present invention as the use of one or more fiducial markers in conjunction with one or more responsive sensors provides improved accuracy in the targeted delivery of protons to positions within a mammalian body, which improved accuracy delimits the unwanted delivery of protons outside of target tissues, and thus the maximum therapeutic benefit is provided when the end point of the proton beam is within the boundary of target tissue.

The invention also relates to a method for the controlled irradiation of a mammalian body, which method includes the steps of:
inserting one or more fiducial markers within a mammalian body, preferably in the locus or near proximity of target tissue present within the said body;
providing a dose of radiation to the said body with a proton probe beam having a proton beam energy level, and thereby causing the emission of a signal or of a material (preferably X-rays) which may be sensed by at least one responsive sensor;
evaluating (e.g., measuring) the sensed signal or material received by the at least one responsive sensor;
comparing the sensed signal or material received by the at least one responsive sensor with a predetermined range of acceptable values of sensed signal or material received;
subsequently, based on the result of the said comparison, delivering a further dose of radiation the said body, optionally after having modified the proton beam energy level modified the proton beam energy level and/or positioning of the proton beam end point to be delivered in the further dose;
and optionally, delivering the further dose to the mammalian body.

The method of the invention may thus be used to provide a series or sequence of dosages of radiation to within a mammalian body, wherein prior to a successive dose to be delivered, the evaluated sensed signal (e.g., X-rays) or material received by the at least one responsive sensor is modified such that the proton beam energy level is greater than or lesser than the proton beam energy level in the immediate prior dose.

Comparison may be performed by a suitably programmed general computer, and the predetermined range of acceptable values of sensed signal or material received (e.g. X-rays) may be present in a machine readable form, e.g. a database or look-up table. The computer may also be used to automatically adjust the operating characteristics of the proton beam delivery system in order to provide a suitable dosage in a subsequent treatment. The computer may be part of a controller unit.

Alternately comparison may be performed by a human operator who may consult a printed, or other readable range of acceptable values of sensed signal or material received (e.g. X-rays) and who may thereafter adjust the operating characteristics of the proton beam delivery system in order to provide a suitable dosage in a subsequent treatment.

The foregoing method may also be practiced upon a non-mammalian body, e.g. a three dimensional space, which may present outside of a mammalian body, or an inanimate “medium”. Accordingly there is also provided a method for the controlled irradiation of an inanimate medium, which method includes the steps of:
inserting one or more fiducial markers within the medium, preferably in the locus or near proximity of a three-dimensional volume or space within the said medium;
providing a dose of radiation to the said medium with a proton probe beam having a proton beam energy level, and thereby causing the emission of a signal or of a material (preferably X-rays) which may be sensed by at least one responsive sensor;
evaluating (e.g., measuring) the sensed signal or material received by the at least one responsive sensor;
comparing the sensed signal or material received by the at least one responsive sensor with a predetermined range of acceptable values of sensed signal or material received;
subsequently, based on the result of the said comparison, delivering a further dose of radiation the said medium, optionally after having modified the proton beam energy level modified the proton beam energy level and/or positioning of the proton beam end point to be delivered in the further dose;
and optionally, delivering the further dose to the medium.

The invention also relates to an apparatus/system useful in providing an improved positioning method for the determination of the controlled delivery of protons within a three dimensional space, which may present outside of a mammalian body. Such is an inanimate “medium”. Consistent therewith, the apparatus/system is used in conjunction with one or more fiducial markers which are inserted or placed within a three-dimensional space/volume, which may be a material, a device or apparatus, or other volume which is not part of a human or mammalian body, viz, the inanimate “medium”. In such an apparatus/system, protons may be delivered to a target locus or target volume present within the (larger) medium within which it is contained. Such an apparatus/system would be operated in a manner similar to (or substantially the same as) that described with reference to the foregoing remarks, and in further reference to the following discussion relating to the various drawing Figures, but would be practiced on a three-dimensional volume which is not part of a human or mammalian body, and the protons would be delivered to the isocenter of target volume present within the larger three-dimensional space, using one or more fiducial markers placed into the medium, which are preferably proximate to the locus of the target locus or target volume present within the larger medium within which it is present.

Certain aspects and preferred embodiments of the present invention are further described with reference to the following drawings. In the following is however to be understood that the disclosed embodiments are merely illustrative and changes can be made into any specific construction or arrangement therein without departing from the principles of the invention. Elements common throughout the drawing figures are labeled throughout using the same numerals and/or letters. Again, it is to be understood that the apparatus, process, and methods of the present invention can be adapted for use in conventional therapeutic methods which utilize a proton beam for the treatment of target tissues within a mammalian body. Such adaptation will be readily understandable to a skilled artisan. In summary, the foregoing description and the following discussion regarding certain specific embodiments is considered as illustrative, and as since numerous modifications and changes will readily occur to those skilled in the art it is not desired to limit the invention to the exact configuration, construction, and method of operation shown and described. Accordingly, all suitable modifications and equivalents may be resorted to, and fall within the scope of the present invention.

Turning now to FIG. 1 therein is depicted in a simple schematic form of a simple embodiment of a proton therapy method and apparatus/system 1 according to the present invention, used to deliver protons to a target tissue within a mammalian body, e.g. a human body. Depicted is a part of a mammalian body 20 (represented by the volume enclosed by a cube with broken lines at the boundary thereof) within which is present a target tissue 30 having had previously embedded within at or near the center thereof (“isocenter”) a fiducial marker 35, here a small gold plate. External of the mammalian body is present a proton source 40, which for example may be a cyclotron or synchrotron for accelerating charged particles such as protons. A beam transport line 42 transports a beam of protons to a beam controller unit 50 which operates to control and direct an output proton beam 41, (having a beam axis represented by arrow “a”) according to the requirements of the desired treatment plan, and directs the controlled output proton beam 41 outwardly therefrom an into the mammalian body 20 and to the locus of the target tissue 30 (or, tumor, or cavity). As is seen in this figure, the end point 44 of the proton beam 41 terminates in the target tissue 30, which is coincident with the Bragg peak 100.

In FIG. 1, it can further be seen that some of the protons which are delivered in the beam of protons 41 impinge upon the embedded fiducial marker 30 and cause the emission of x-rays therefrom. The relative position of the fiducial marker 35 relative to the boundary 31 of the target tissue 30 may be previously established, and correlated to the common three-dimensional x-y-z set of axes, which is shown in the figure. The X-rays emitted along a path 32 and through two oriented variable masks 62, 64 having slits 63, 65 are received by an x-ray sensor 61 which may be a plate or an array, which element comprise an embodiment of a responsive sensor 60 device. The output of the x-ray sensor 61 provides information and/or data concerning the nature of x-rays emitted from the fiducial marker 35 and the position of the endpoint 44 of the proton beam 41, which position may also be correlated in three-dimensions according to the common three-dimensional x-y-z set of axes. Thus, information/data regarding the x-ray signals sensed by the responsive sensor 60 may be used to evaluate the location of the proton beam 41 relative to the position of the target tissue 30, and the position of the endpoint 44 of the proton beam 41 (and the physical position of the Bragg peak 100 within the mammalian body 20) can be determined as well. The information/data can be used to further adjust or modify the further operation of the proton source 40 and/or the operation of the beam controller unit 50 in the current, or subsequent treatment. For example the size (e.g, cross-sectional area) of the proton beam 41 may be adjusted to more closely conform to the contours of the boundary 31 of the target tissue in a plane perpendicular to the beam axis “a” of the output proton beam 41, and/or the direction of the output proton beam 41 can be altered (e.g, wherein the output proton beam 41 is of the “scanning” type), and/or the location of the position of the endpoint 44 of the proton beam 41 can be adjusted as well. Such control may be performed by modifying the performance characteristics of one or both of the proton source 40 and/or the beam controller unit 50. Such adjustments modify the dosage of protons being delivered to the mammalian body. Such adjustments may be entered manually, or may be effectuated via the use of a computer/controller 70 as discussed in the following FIG. 2.

The proton therapy method and apparatus/system 1 disclosed on FIG. 2 includes many of the same elements disclosed on FIG. 1. In this figure, the proton source 40 is not shown but further details are illustrated concerning the beam controller unit 50 which operates to control and direct an output proton beam 41, here a wider “pencil type” beam having a cross-section represented by the intersecting plane sections “b” which are perpendicular to the beam axis represented by arrow “a”. Protons “b” within the output proton beam 41 move in the direction of the arrow “a”. The beam controller unit 50 here comprises a first scanning magnet 51 for modifying the direction of the output proton beam 41 in a first direction (y), perpendicular to the main beam axis (x) and a second scanning a magnet 52 for modifying the direction of the output proton beam 41 in a second direction (z), perpendicular to the main beam axis (x) direction. Additionally, a beam position monitor 53 may also present in is provided for measuring the (y,z) position of the beam, perpendicular to the main beam axis (z). Reference is made to the reference x-y-z axis illustrated in the Figure. Additionally, a dose detector 54 may also be present, which operates to measure the dose of protons “P” delivered to the target tissue 35 by the controlled, output proton beam 41 traversing it, and/or impinging upon it. As before, the beam controller unit 50 operates to fulfill the requirements of the desired treatment plan, and directs the controlled output proton beam 41 outwardly therefrom an into the mammalian body 20 and to the locus of the target tissue 30 (or, tumor, or cavity). As is seen in this figure, the end point 44 of the proton beam 41 terminates in the target tissue 30, which is coincident with the Bragg peak 100.

In the embodiment of FIG. 2 is illustrated a fiducial marker 35 implanted within the interior of the target tissue 30, and preferably near the isocenter thereof as is illustrated. In additional, further fiducial marker 35′ is also implanted, here adjacent to, or at the boundary 31 of the target tissue 30. In the apparatus/system of FIG. 2, there is present both a first responsive sensor 60 device which has features substantially similar to those described reference to FIG. 1. This first responsive sensor 60 device is physically orientated such that the X-rays emanating from the first fiducial marker 35 and emitted along a path 32 and through two oriented variable masks 62, 64 having slits 63, 65 are received by the x-ray sensor 61 of the first responsive sensor device 60. A second responsive sensor device 60′ is also present, and is physically orientated such that the X-rays emanating from the second fiducial marker 35′ and emitted along a path 32′ and through two oriented variable masks 62′, 64′ having slits 63′, 65′ are received by the x-ray sensor 61′ of the second responsive sensor device 60′. As the physical distance between the first fiducial marker 35 and the second fiducial marker 35′, and their orientation relative to the target tissue 30 is known (as may be ascertained with reference to the reference axes x-y-z, the signals/particles received by the first responsive sensor 60 and second responsive sensor 60′ can be evaluated, and from which may be determined the location of the endpoint 44 of the output proton beam 41. If necessary, the operating characteristics of the beam controller unit 50 can be modified in order to adjust its performance characteristics when delivering a subsequent dosage of protons P. In the embodiment according to FIG. 2, information and/or data concerning the nature of x-rays emitted from the fiducial markers 35, 35′ and the position of the endpoint 44 of the proton beam 41 is supplied via communication path 71 (e.g, one or more wires, cable or computer bus, or wireless (i.e., Bluetooth, WiFi) or optical data path) are in data communication with a controller/computer 70 which processes said information and/or data and outputs further control signals via a further communication path 72 ((e.g, one or more wires, cable or computer bus, or wireless (i.e., Bluetooth, WiFi) or optical data path) as inputs to the beam controller unit 50 which may modify the operating characteristics thereof, especially to adjust the proton beam 41 correspondingly so that it targets the delivery of protons P to the locus (or volume) of the target tissue 3, and preferably such that minimal energy from the protons is discharged beyond the endpoint 44 of the proton beam 41 and to surrounding non-target tissue within the mammalian body.

FIG. 3 depicts a further embodiment of a proton therapy method and apparatus/system 1 which includes many of the same elements disclosed on FIGS. 1 and 2. Here the beam controller unit 50 operates to control and direct an output proton beam 41 in a scanning manner, viz., wherein a narrower proton beam 41 is diverted during operation to move in directions y and/or z away from the beam axis “a” (which coincides with reference axis x) in order to provide a wider beam. The movement of the beam may be controlled to more closely match the profile/boundary 31 of the target tissue, in a plane perpendicular to the beam axis “a”. Such may be used to case the proton beam 41 to scan in two directions in a plane perpendicular to the beam axis when delivering a dose of protons to the mammalian body 20 and especially to the locus of the target tissue 30. A cross-section of the proton beam 41 us are represented by the intersecting plane sections “b”, protons “P” within the output proton beam 41 move in the direction of the arrow “a”. Scanning, and the dosage of the protons delivered is controlled by one or more of the proton source 40 and/or the beam controller unit 50. In this figure, it is to be noted that there are present three fidicucial markers 35, 35′ and 35″ each of which is embedded within the mammalian body 20 in the locus of the target tissue 30, but each of the fiducial markers 35, 35′ and 35″ are positioned outside of the boundary 31 of the target tissue 30. The placed fiducial markers 35, 35′ and 35″ with reference to a reference three-dimension axes z-y-z (also depicted) with reference to one another, and with reference to the volume of the target tissue and its position can be determined by conventional techniques, e.g, radiography, MRI, CAT or other. Reference is made to the reference x-y-z axis illustrated in the Figure.

Also present is a first responsive sensor 60 device, a second responsive sensor 60′ device and a third responsive sensor 60″ device each of which are substantially similar to those described reference to FIGS. 1 and 2. Each of the responsive sensor 60, 60′ and 60″ devices are physically orientated such that the X-rays emanating from one of the first fiducial markers 35, 35′ and 35″ and emitted along a corresponding path 32, 32′ and 32″ are received by a corresponding responsive sensor 60, 60′ and/or 60″ device. As the physical distance between the first fiducial marker 35, and the second fiducial marker 35′ and the third fiducial marker 35′, and their orientations relative to the target tissue 30 are known (as may be ascertained with reference to the reference axes x-y-z,) the signals/particles received by each of the responsive sensors 60, 60′ and 60″ can be evaluated, and from which may be determined the location of the endpoint 44 of the output proton beam 41. Preferably the endpoint 44 corresponds to the Bragg peak 100 of the proton beam 41 as well. If necessary, the operating characteristics of the beam controller unit 50 can be modified in order to adjust its performance characteristics when delivering a subsequent dosage of protons P. In the embodiment according to FIG. 3, information and/or data concerning the nature of x-rays emitted from the fiducial 35, 35′, and the position of the endpoint 44 of the proton beam 41 is supplied via communication path 71 (e.g, one or more wires, cable or computer bus, or wireless (Bluetooth, WiFi) or optical data path) are in data communication with a controller/computer 70 which processes said information and/or data and outputs further control signals via a further communication path 72 ((e.g, one or more wires, cable or computer bus, or wireless (Bluetooth, WiFi) or optical data path) as inputs to the beam controller unit 50 which may modify the operating characteristics thereof, especially to adjust the proton beam 41 correspondingly so that it targets the delivery of protons P to the locus (or volume) of the target tissue 30, and preferably such that minimal energy from the protons is discharged beyond the endpoint 44 of the proton beam 41 and to surrounding non-target tissue within the mammalian body. As before, the beam controller unit 50 operates to fulfill the requirements of the desired treatment plan, and directs the controlled output proton beam 41 outwardly therefrom an into the mammalian body 20 and to the locus of the target tissue 30 (or, tumor, or cavity). As is seen in this figure, the end point 44 of the proton beam 41 terminates in the target tissue 30, which is coincident with the Bragg peak 100.

FIG. 1 illustrates an embodiment wherein the fiducial marker 35 is positioned within the isocenter of the target tissue 30. FIG. 2 illustrates an embodiment wherein at least one fiducial marker 35′ is placed at or near the boundary 31 of the target tissue 30. FIG. 3 illustrates an embodiment wherein fiducial markers 35, 35′ and 35″ are placed in the locus of the target tissue 30, but outside of the boundary 31 of the target tissue 30. These embodiments are not meant to be limiting, but illustrative. This clearly contemplated that in any of the methods or systems according to the invention, one or more fiducial markers may be placed at any of the one or more foregoing locations. In the embodiment of FIG. 1 a single responsive sensor 60 is present.

FIG. 2 illustrates an embodiment where two responsive sensors 60, 60′ are present and two fiducial markers 35, 35′ are also present. FIG. 3 illustrates an embodiment wherein three responsive sensors 60, 60′ and 60″ are present, and wherein three fiducial markers 35, 35′ and 35″ are also present. Whereas each of the embodiments illustrated depicts a single responsive sensor 60 matched or responsive to a single fiducial marker, such is not necessary and it is contemplated that suitable responsive sensors include those which may be responsive to two or more individual fiducial markers.

FIGS. 4A, 4B, 4C and 4D illustrate certain embodiments of preferred fiducial markers 35 of various different configurations.

In FIG. 4A, a plurality of individual fiducial markers 35 herein the shape of spheres 35A are illustrated. The spheres 35A may be hollow, or may be solid. The spheres may be coated or uncoated. The spheres 35A may be of the same or of different sizes. A single sphere 35A, or a plurality of spheres 35A may be used as a fiducial marker. Advantageously the spheres have a diameter in the range of about 100-1000 microns, preferably about 300-600 microns.

FIG. 4B depicts a plurality of individual fiducial markers 35 herein the shape of flat plates or curved plates 35C. The plates 35C may be generally flat and or curved, as depicted, and each may be coated or uncoated. The plates 35C may be of the same or of different sizes. A single plate 35C, or a plurality of plates 35C may be used as a fiducial marker. Advantageously the plates 35C have a width dimension of W2 in the range of about 300-1200 microns, preferably about 500-900 microns, and a length L2 dimension in the range of about 500-2200 microns, preferably about 800-1500 microns, and a thickness dimension in the range of about 50-750 microns, preferably about 200-400 microns. When curved, the inner radius of curvature of a curved plate 35C has a radius dimension in the range of about 200-4000 microns, preferably about 600-2000 microns.

In FIG. 4C, are depicted a plurality of individual fiducial markers 35 herein the shape of rods 35B. The rods 35B may be hollow, or may be solid, and they may be coated or uncoated. The rods 35B may be of the same or of different sizes. Each rod 35B may have a circular or non-circular cross-second along its length L1. A single rod 35B, or a plurality of rods 35B may be used as a fiducial marker. Advantageously the rods 35B have a diameter D1 in the range of about 500-1800 microns, preferably in the range of about 800-1200 microns, and a length L1 in the range of about 300-3000 microns, preferably about 600-2000 microns.

In FIG. 4D is illustrated a preferred embodiment of a fiducial marker 35, here in the shape of a helix. Advantageously, the helix is formed of a wire of a suitable material, especially gold, a gold alloy, or other suitable material. Preferably, the helix has an overall length L3 of about 1000-10000 microns, preferably about 3000-5000 microns, a diameter of about 100-1200 microns, preferably about 500-800 microns, and the diameter D4 of the wire is about 50-350 microns, preferably about 100-200 microns.

FIG. 4E depicts a further embodiment of a fiducial marker 35, here in the shape of a circle, ring or torus. Advantageously, the circle, ring or torus has an overall thickness T1 of about 50-700 microns, preferably about 200-400 microns, an outside radius R2 of about 500-4000 microns, preferably about 1000-2000 microns, and an inside radius R1 of about 300-1200 microns, preferably about 600-800 microns.

1. A positioning method for the determination of the delivery of protons within a mammalian body or an inanimate medium, which method comprises the steps of:
determining at least one operating characteristic of a proton beam entering a mammalian body or inanimate medium by sensing the release of particles and/or a signal from at least one fiducial marker located within.

2. The positioning method of claim 1, wherein the release of particles and/or a signal from two or more fiducial markers located within the mammalian body or inanimate medium is sensed.

3. The positioning method of claim 1, wherein the release of particles and/or a signal is a release of photons.

4. The positioning method of claim 2, wherein the release of particles and/or a signal is a release of photons.

5. The positioning method of claim 1, wherein the operating characteristic determined is the end point of the proton beam.

6. The positioning method of claim 1, wherein the operating characteristic determined is the end point of the proton beam within the mammalian body or inanimate medium.

7. The positioning method according to claim 1, wherein the method includes the further step of:
subsequent to determining at least one operating characteristic, modifying the subsequent delivery of protons to the mammalian body.

8. The positioning method according to claim 1, wherein the fiducial marker is embedded within a mammalian body and is at a boundary of, or in the locus of a target tissue.

9. The positioning method according to claim 1, wherein the fiducial marker emits x-rays in response to being contacted by the proton beam.

10. The positioning method according to claim 1, wherein the fiducial marker comprises one or more of: gold, gold alloys, platinum, silver palladium, zirconium, molebdenum, and combinations or mixtures thereof.

11. A method for the controlled irradiation of a mammalian body, which method includes the steps of:
a method for the controlled irradiation of a mammalian body, which method includes the steps of:
inserting one or more fiducial markers within a mammalian body, preferably in the locus or near proximity of target tissue present within the said body;
providing a dose of radiation to the said body with a proton probe beam having a proton beam energy level, and thereby causing the emission of a signal or of a which may be sensed by at least one responsive sensor;
evaluating the sensed signal or material received by the at least one responsive sensor;
comparing the sensed signal or material received by the at least one responsive sensor with a predetermined range of acceptable values of sensed signal or material received;
subsequently, based on the result of the said comparison, delivering a further dose of radiation the said body, optionally after having modified the proton beam energy level modified the proton beam energy level and/or positioning of the proton beam end point to be delivered in the further dose;
and optionally, delivering the further dose to the mammalian body.

12. A method for the controlled irradiation of an inanimate medium, which method includes the steps of:
inserting one or more fiducial markers within the medium, preferably in the locus or near proximity of a three-dimensional volume or space within the said medium;
providing a dose of radiation to the said medium with a proton probe beam having a proton beam energy level, and thereby causing the emission of a signal or of a material which may be sensed by at least one responsive sensor;
evaluating the sensed signal or material received by the at least one responsive sensor;
comparing the sensed signal or material received by the at least one responsive sensor with a predetermined range of acceptable values of sensed signal or material received;
subsequently, based on the result of the said comparison, delivering a further dose of radiation the said medium, optionally after having modified the proton beam energy level modified the proton beam energy level and/or positioning of the proton beam end point to be delivered in the further dose;
and optionally, delivering the further dose to the medium.

13. A system which delivers protons to within a mammalian body or an inanimate medium, which system includes:
a fiducial marker,
a responsive sensor, which is senses or receives a signal or particle which is emitted from the fiducial marker when the fiducial marker is impinged by protons from a proton beam when the fiducial marker is positioned within a mammalian body;
a proton beam source, and,
a controller unit which operates to control and direct an output proton beam from the proton beam source and to a mammalian body.

14. A system according to claim 13, which includes:
a plurality of responsive sensors.

15. A system according to claim 13, which includes:
a plurality of fiducial markers.

16. A system according to claim 13, wherein:
the controller unit operates, in response to data collected from each responsive sensor may be used to validate the position of the end point of the proton beam, and may be used to adjust the position of the proton beam entering the mammalian body so that the delivery to target tissue within the mammalian body is optimized.

17. A system according to claim 13, wherein the responsive sensor senses X-rays or photons.

18. A system according to claim 14, wherein the responsive sensors sense X-rays or photons.
